(19) United States
(12) Patent Application Publication
(10) Pub. No.: US 2007/0055172 A1
(43) Pub. Date: Mar. 8, 2007

(54) Multi Forceps Biopsy Catheter

(76) Inventor: Nitesh Ratnakar, Oak Creek, WI (US)

Correspondence Address: NITESH RATNAKAR, ROUTE 3,, BOX 179-A, ELKINS, WV, 26241, US

(21) Appl. No.: 11/162,269

(22) Filed: Sep. 4, 2005

(51) Int. Cl. A61B 10/00; A61B 17/00

(52) U.S. Cl. 600/564; 600/562; 606/205

(57) ABSTRACT

The present invention relates to biopsy catheters; more specifically to a biopsy catheter with means to obtain multiple tissue specimens during a single operation of the said biopsy catheter. It comprises of a proximal handle from which extends a shaft; the said shaft housing multiple forceps biopsy assemblies at pre-determined intervals along its length; each forceps biopsy assembly comprising of a proximal housing; on which two serrated jaws are disposed; each jaw mated with other about a clevis pin, which is cast unitarily with a clevis that extends into the housing; the housing being attached to the shaft by means of a hinge joint. Each jaw extends proximally and terminates in a tang, each tang arranged so as to receive a control pull wire there through. Each control pull wire from the tang flexibly extends into the corresponding main control pull wire, the main control pull wire extending through the bore in the shaft into the handle; where it is operatively engaged to a spool. Distal and proximal movement of the spool respectively opens and closes the jaws of all forceps biopsy assemblies disposed on the shaft. The outer and inner side of the housing of each forceps biopsy assembly is attached to a deployment pull wire; each deployment pull wire flexibly extending into the corresponding main deployment pull wire extending through a bore in the shaft of the biopsy catheter into the handle; and thereon to a “deployment actuator”. Operation of the “deployment actuator” deploys the forceps biopsy assemblies placed along the shaft by moving the forceps biopsy assemblies sideways, away and perpendicular to the shaft. The biopsy catheter of the present invention provides the user means to obtain multiple biopsies from different parts of an organ during a single operation of the said biopsy catheter.

### FIELD OF INVENTION

The present invention relates to biopsy catheters, more specifically to a biopsy catheter wherein means are provided to take multiple tissue specimens from different sites of an organ during single operation of the said biopsy catheter.

### PRIOR ART

A number of prior art devices relating to biopsy catheters, more specifically relating to biopsy catheters used in conjunction with endoscopic procedures have been described. In U.S. Pat. No. 5,666,965 issued on Sep. 16, 1997; Bales et al have described a biological forceps device for the taking of tissue samples from a body, comprising a flexible main coil attached at its distal end to a pair of homologous cast jaws. The jaws have radially arranged teeth on their distal most end. The jaws are opened and closed by attachment to a pair of pull wires which extend through the main coil, into a handle at its proximal end. The handle has a spool which slides about a central shaft attached to the main coil. The spool is attached to the pull wires, so that movement of the spool with respect to the central shaft, effectuates a force on the proximal ends of the levered jaws, to open and close them, appropriately.

In U.S. Pat. No. 4,785,825 issued on Nov. 22, 1985; Romaniuk et al have described a safety biopsy forceps, in particular for infants, comprising of operating forceps, a flexible shaft, a pulling element, as well as a head with cutting- and/or grasping elements, of which at least one element is moveable by means of the pulling element, provided with a head with cutting- and/or grasping element which is detachably fastened on the flexible shaft. The flexible shaft is detachably fastened to the operating forceps and the pulling element to the operating forceps. The pulling element is adjustable for the setting of the cutting- and/or grasping elements and the setting of the force of the cutting process and can also be locked in after the adjustment. The cutting- and/or grasping elements are provided at the joint with a recess which prevents tissue from becoming lodge during closing of the elements.

In U.S. Pat. No. 4,815,476 issued on Mar. 29, 1989; Clossick has described a biopsy forceps device comprising of a handle portion an elongate flexible hollow body portion having a proximal end coupled to the handle portion and a distal end. A forceps assembly is coupled to the distal end and includes a pair of forceps. A stylet control wire in the body portion is coupled to the pair of forceps at the distal end of the body portion. A locking hub assembly is coupled between the handle portion and the proximal end of the body around the stylet/control wire and includes a locking hub and locking means for locking the stylet/control wire in an axial position thereof to the locking hub assembly relative to the body portion upon rotation of the locking hub.

Operation: Generally speaking, when a tissue specimen has to be obtained during an endoscopic procedure, the biopsy catheter is passed through the instrument channel of the endoscope. The tip of the endoscope is then maneuvered to align the jaws of the biopsy forceps assembly with the tissue to be sampled. The jaws of the forceps assembly are then opened using the actuator assembly, subsequent to which the jaws are brought in opposition to the tissue to be sampled. The jaws are then closed firmly using the actuator assembly; whereby a piece of the desired tissue is grasped firmly between the jaws. The biopsy catheter is then pulled away from the tissue surface. This maneuver severs the grasped tissue from its parent organ. At this time, the jaws are maintained in the closed position and the biopsy catheter is pulled out of the endoscope. The tissue sample caught between the jaws is then retrieved.

A major drawback of the present biopsy catheter is that it allows only one tissue sample to be obtained during a single operation of the said catheter. When there is a need to obtain multiple tissue specimens, the biopsy catheter has to be passed multiple times through the endoscope. This makes the endoscopic procedure labor intensive and time consuming and increases the sedation/anesthesia time, consequently leading to increased adverse outcomes. Two common conditions that require multiple biopsies during endoscopic examination are i) Barrett's esophagus; and ii) Inflammatory Bowel Disease. Both these conditions increase the risk of developing cancer of the gastrointestinal tract. Frequent endoscopic examination and multiple biopsy specimens at each endoscopic examination are needed to thoroughly evaluate for pre malignant or early malignant lesions. In case of Barrett's esophagus, it is recommended that endoscopic examination be done regularly; and four quadrant tissue specimens be taken at every 1 cm throughout the length of the Barrett's esophagus. This is labor intensive because the endoscopist has to pass the biopsy catheter multiple times through the endoscope in order to obtain adequate number of tissue specimens. Moreover, it is frequently difficult to obtain latter tissue specimens as the field of operation becomes bloody and difficult to visualize as a result of trauma induced by prior biopsies. The end result is that often the endoscopist is unable to obtain adequate number of tissue specimens; and pre malignant and early malignant lesions are frequently missed.

It is even more problematic to take adequate number of biopsy specimens in patients with Inflammatory Bowel Disease; where it is recommended that endoscopic examination of the colon be done every year and that four quadrant tissue specimens be taken at every 10 cm during each such examination. Considering that average length of a colon is 100 to 150 cm; 40-60 biopsy specimens need to be taken at each endoscopic examination to adequately examine for pre malignant and early malignant lesions. As is evident, this makes the endoscopic examination of the colon cumbersome and time consuming; as a conventional biopsy catheter needs to be advanced through the endoscope 40-60 times in order to obtain adequate number of tissue specimens. In addition, it is very difficult to accurately measure distances during endoscopic examination, which makes it very difficult to be certain that tissue specimens have been obtained every 10 cm as recommended. Because of the aforementioned, it common for inadequate number of tissue specimens to be obtained during surveillance endoscopic examination of the colon for Inflammatory Bowel Disease.

### OBJECTS OF THE INVENTION

Accordingly the object of the present invention is to present a biopsy catheter with means to obtain multiple tissue specimens from different sites of an organ during a single operation of the said catheter.

### SUMMARY OF THE INVENTION

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods and systems for carrying out one or several purposes of the present invention. It is important, therefore, that the claims be regarded as including such equivalent constructions insofar as they do not depart from the spirit and scope of the present invention.

The present invention presents a biopsy catheter; hereby referred to as ‘multi forceps biopsy catheter’, wherein multiple forceps biopsy assemblies are disposed along the length of its shaft; and wherein means are provided to deploy the forceps biopsy assemblies sideways from the shaft. Presence of multiple forceps biopsy assemblies enables multiple tissue specimens to be obtained during single operation of the ‘multi forceps biopsy catheter’. The construction and operation of the ‘multi forceps biopsy catheter’ is outlined in the following paragraphs. The ‘multi forceps biopsy catheter’ has three main parts—forceps biopsy assembly, handle and shaft.

The forceps biopsy assembly comprises of a pair of jaws movably attached to a housing; each jaw of the pair is a duplicate of the other jaw. Each jaw is somewhat hemi spherically shaped having an elongated portion, which extends proximally into a tang. Each jaw has a generally U-shaped distal most end on which is defined a plurality of radially disposed teeth. The teeth on one side of the longitudinal centerline of the jaw are displaced by one-half pitch from the corresponding teeth on the other side of the longitudinal centerline on that jaw. The displacement by one-half pitch of the teeth on one side of the jaw relative to those corresponding teeth on the other longitudinal side of the jaw permits tight interlocking of the radial teeth of the two jaws when the two jaws are in closed position. Each jaw extends proximally and terminates in a tang; each tang having a hole so as to receive a control pull wire there through. The ‘control pull wires’ from the tangs flexibly joins the ‘main control pull wire’ in the bore of the shaft which extends into the handle; and is thereafter operatively engaged to a spool located on the handle. Each jaw is mated with one another about a clevis pin, which is cast unitarily with a clevis. The clevis extends into a housing, which is attached to the shaft by means of a hinge joint or other suitable mechanical articulation. Two ‘deployment pull wires’ flexibly extends from opposite surfaces of the housing and joins the main deployment pull wire in the bore of the shaft which extends into the handle; and thereafter to a “deployment actuator” assembly. The ‘deployment pull wires’ are arranged in a way to enable deployment and retraction of the housing perpendicular to the axis of the shaft.

The shaft extends from the distal forceps biopsy assembly to the handle. It is preferably made of a flexible and strong material to enable it to navigate the turns of gastrointestinal tract. A support base is provided, which extends along the base of the shaft. The support base is intended to provide extra support to the forceps biopsy assemblies placed along the shaft. Two bores extend throughout the length of the shaft, the proximal end of which are in continuity with the bore of the handle. The said bores house the main deployment pull wires from the housings and the main control pull wires from the jaws of the biopsy forceps assemblies. In the ‘multi forceps biopsy catheter’ of the present invention; there are multiple joggled ‘control pull wires’ and ‘deployment pull wires’ coming from a multitude of forceps biopsy assemblies. The diameter of the bores of the shaft and of the central shaft of the handle is large enough to accommodate the required number of control and deployment pull wires required in the construction and operation of the ‘multi forceps biopsy catheter’.

The handle comprises a central shaft about which a displaceable spool is disposed. The central shaft has a longitudinally directed stepped diameter bore extending therein on its distal end, and a ‘deployment actuator’ and a thumb ring on its proximal most end. The proximal end of the shaft extends into the bore on the central shaft. The bore in the central shaft of the handle has a stepped configuration. The distal end of the bore having a slightly larger diameter than the second or intermediate bore, or the third or proximal end of the bore in the central shaft. A locking coil is arranged to mate within the stepped large outer diameter (distal end) of the central shaft. The diameter of the locking coil is slightly smaller than the outer diameter of the shaft. The shaft is screwed into the locking coil disposed within the central shaft. A sheath, which acts as a strain relief, is disposed distally of the locking coil about the shaft within the central shaft. The sheath holds the locking coil within the first stepped bore in the central shaft. The strain relief is bonded to the bore of the central shaft. The proximal end of the ‘control pull wires’ extend through the proximal end of the shaft and into a thin anti-kink tube in the narrowest third stepped bore in the central shaft. The cross pin fits through a slot at the midpoint of the central shaft. The slot is in communication with the third bore therein. A cross pin mates with the slot across the central shaft. The proximal most end of the ‘control pull wires’ are locked into an opening in the cross pin. The ends of the cross pin mate with slots in the spool so as to facilitate corresponding motion in the main ‘control pull wires’. The main “deployment pull wires” pass through the bore of the central shaft of the handle onto the ‘deployment actuator’. The ‘deployment actuator’ in the preferred embodiment is a wheel to which the main ‘deployment pull wires’ are operatively engaged.

Operation: Proximal movement of the spool with respect to the central shaft effectuates a pull on the ‘control pull wires’ so as to create a pivotable motion of the tangs on the proximal end of the jaws, to cause the jaws to engage to one another. Movement of the spool distally with respect to the central shaft effectuates a compression on the pull wire thus causing arcuate movement of the tangs on the proximal end of each jaw to force a pivoting motion about the clevis pin thus opening the respective jaws. Because of the extension of ‘control pull wires’ from all forceps biopsy assemblies to the main ‘control pull wires’; proximal and distal movement of the spool results in closing and opening of the jaws of all forceps assemblies present on the ‘multi forceps biopsy catheter’. Clock wise and counter clock movement of the ‘deployment actuator’ effects the deployment and retraction of forceps biopsy assemblies away from and perpendicular to the shaft. Because of the extension of the ‘deployment pull wires’ from all forceps biopsy modules into the main ‘deployment pull wire’; operation of the ‘deployment actuator’ effects the deployment and retraction of all forceps biopsy assemblies along the length of the shaft.

### BRIEF DESCRIPTION OF THE DRAWINGS

The objects and advantages of the present invention will become more apparent when viewed in conjunction with the following drawings, in which:

FIG. 1A is a side view of the multi forceps biopsy catheter.

FIG. 1B is a side view of the handle of the multi forceps biopsy catheter.

FIG. 2A is a side elevational view of the distal most end of the multi forceps biopsy catheter with a needle, with its cutter jaws being opened.

FIG. 2B is a plan view, partly in section, of the distal end of the multi forceps biopsy assembly, with a needle.

FIG. 2C is a side elevational view partly in section, of the multi forceps biopsy catheter shown in FIG. 2A.

FIG. 3 is a cross section view of the shaft of the multi forceps biopsy catheter.

FIG. 4A is a side view of the multi forceps biopsy catheter showing the construction of the forceps biopsy assembly with respect to the shaft.

FIG. 4B is a side view of the multi forceps biopsy catheter of FIG. 4A wherein the forceps biopsy assembly and jaws contained therein are in a deployed position.

FIG. 5A is a side view of the multi forceps biopsy catheter comprising of multiple forceps biopsy assemblies wherein the forceps biopsy assemblies are in a deployed position.

FIG. 5B is a side view of the multi forceps biopsy catheter comprising of multiple forceps biopsy assemblies wherein jaws contained in the said forceps biopsy assemblies are in a deployed position.

FIGS. 6A& 6B is a side view showing the construction of the handle of the multi forceps biopsy catheter.

### DETAILED DESCRIPTION OF THE PREFERRED EMBODIMENT

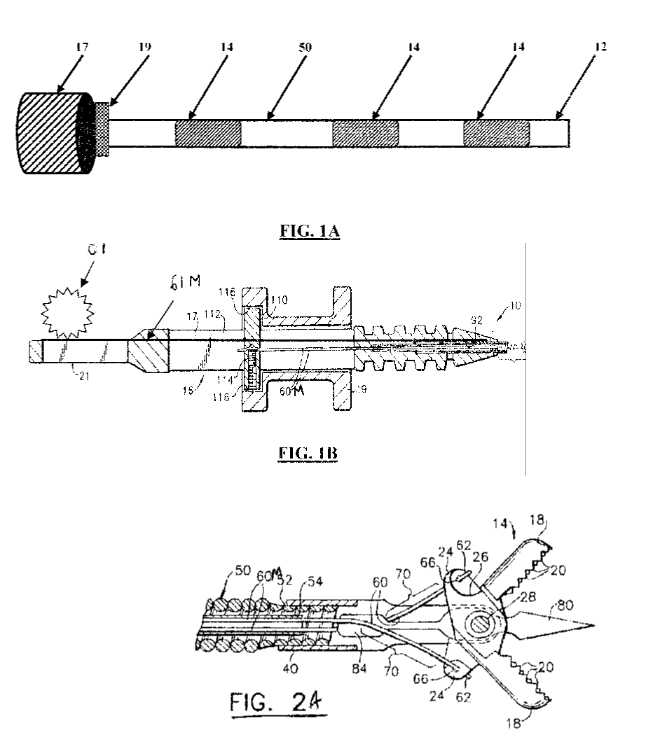

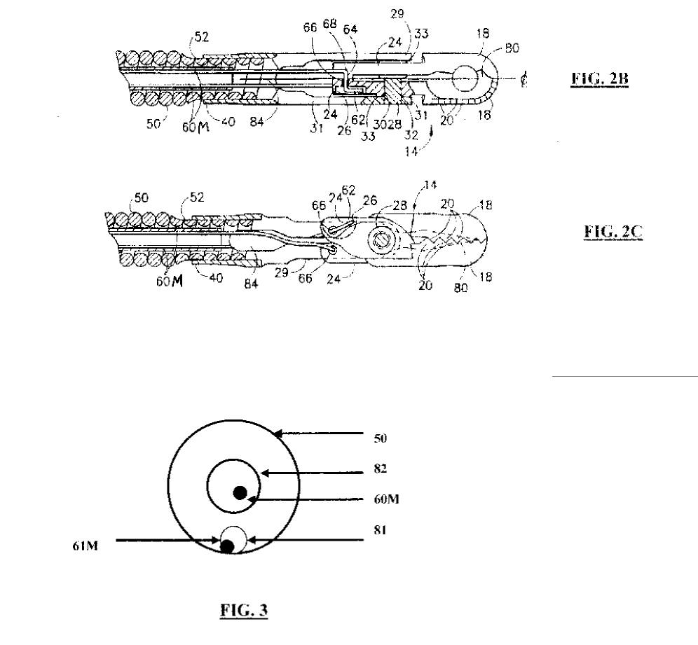

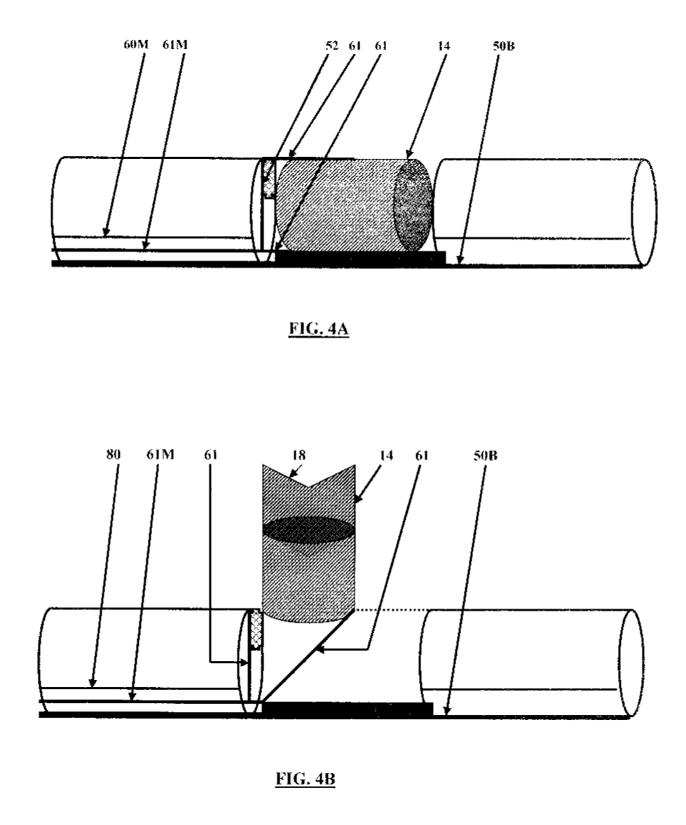

Referring now to the drawings in detail and particularly to FIGS. 1A & 1B, there is shown a ‘multi forceps biopsy catheter’ 10, having 1) a distal end 12; 2) a proximal end 16 comprising of handle 17, spool 19, thumb ring 21 and deployment actuator 01; and 3) a shaft 50 extending from the distal end 12 to the proximal end 16; housing multiple forceps biopsy assemblies 14. Each forceps biopsy assembly 14 comprises a pair of jaws 18, each of which is a duplicate of the other. Each jaw 18, as may be seen in FIG. 2A, is a generally elongated somewhat hemi spherically shaped structure having a distal most end and a proximal most end. Each jaw 18 has on its distal most end, an array of teeth 20 generally radially directed about a point “R”. Each jaw 18 has a generally longitudinal centerline. The teeth 20 on one side of the longitudinal centerline of each jaw 18 being displaced by one half pitch from the corresponding teeth 20 on the other side of the longitudinal centerline on that jaw 18. The displacement by one half pitch by the teeth on one side of the jaw 18 is relative to those corresponding teeth 20 on the other longitudinal side of the jaw 18 permits the jaws to automatically mate and effectuate proper alignment there between when they close onto one another. Each jaw 18 has a proximal most end, which comprises a tang 24. Each tang 24 has a generally semicircular recess position 26 on its outer side thereof. A bore 30 extends transversely through the midpoint between the distal and proximal most ends of each jaw 18. Each jaw 18 is mated with one another and so as to each be levered about a clevis pin 28 which extends through the bore 30 on each respective jaw 18. Each jaw 18 has an annular boss 33 disposed about the outer face of its bore 30, as shown in FIG. 2B. The boss 33 acts as a bearing surface to reduce friction. The clevis pin 28 is received in a hole 32 in clevis 34 as shown in FIG. 2B. The clevis 34 extends proximally into a housing 40. The clevis 34, the housing 40 and clevis pin 28 are made from a common casting. A control pull wire 60 extends from each tang 24 to the corresponding main control pull wire 60M; wherein each main control pull wire 60M receives ‘control pull wires’ 60 from similarly aligned tangs 24 in order to effect concurrent movement of similarly aligned tangs 24 of all forceps biopsy assemblies 14 of the ‘multi forceps biopsy catheter’. The main ‘control pull wire’ 60M extends proximally into the handle 17 and is thereafter operationally connected to the spool 19, as discussed subsequently. The distal most end of each control pull wire 60 has a Z-bend therein. The Z-bend of each pull wire 60 has a first portion 62, which is rotatably disposed in the recess 26 in the tang 24 of each cutter jaw 18. The Z-bend has a second portion 64, which extends through a bore 66 in the proximal most end of the tang 24, as best shown in FIGS. 2A &2B. A 90° bend 68 (FIG. 2B) between the second portion 64 and the control pull wire 60 eliminates any pinching. Each control pull wire 60 has a reflex curve 70 as shown in FIG. 2A, extending between their distal most ends and the distal most end of the shaft 50. The reflex curve 70 helps to open the jaws 18 when the spool 19 on the handle 17 is displaced distally thereto. Each forceps biopsy assembly 14 has a needle 80 disposed between the jaws 18 as shown in FIG. 2A. The needle 80 helps stabilize the tissue during biopsy and also helps keep the biopsied tissue within the jaws once it is severed from its parent organ.

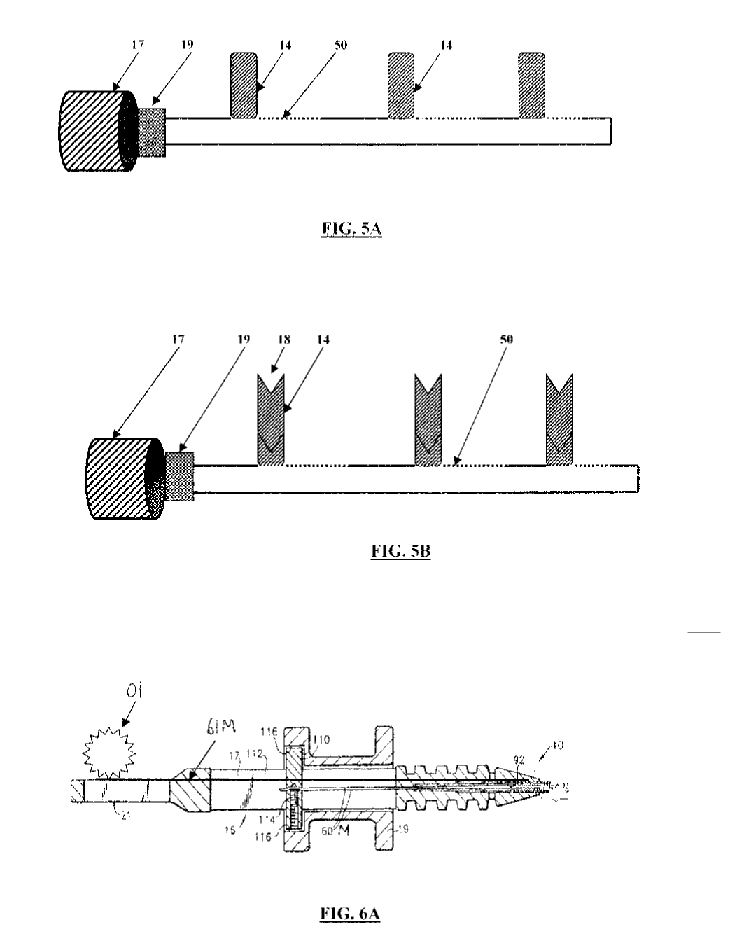

The construction of the shaft 50 is shown in FIGS. 3, 4A & 4B. It is preferably made of a strong but flexible material to enable it to maneuver the turns of the gastrointestinal tract. As shown in FIG. 3, the shaft 50 has a central bore 82 extending from it distal end to its proximal end; the said bore 82 accommodating the main ‘control pull wires’ 60M; and a peripheral bore 81 which accommodates the main ‘deployment pull wires’ 61M. The shaft 50 is supported with a support base 50B (FIGS. 4A & 4B) throughout its length. Multiple forceps biopsy assemblies 14 are placed at pre determined intervals along the length of the shaft 50, as shown in FIGS. 4A, 4B, 5A & 5B. The housing of each forceps biopsy assembly 14 is attached to the shaft 50 by means of a hinge joint 52 or any other suitable mechanical articulation. Each forceps biopsy assembly 14 has a pair of ‘deployment pull wires’ 61 that extends from the outer and inner sides of the housing to the main ‘deployment pull wire’ 61M in the bore 81 of the shaft 50. The main deployment pull wire 61M is attached proximally to a deployment actuator 01, the operation of which applies/relieves tension on the ‘deployment pull wires’ 61M & 61; thereby deploying/retracting the corresponding forceps biopsy assemblies 14. This is illustrated in FIG. 5A where the forceps biopsy assemblies are in a deployed position.

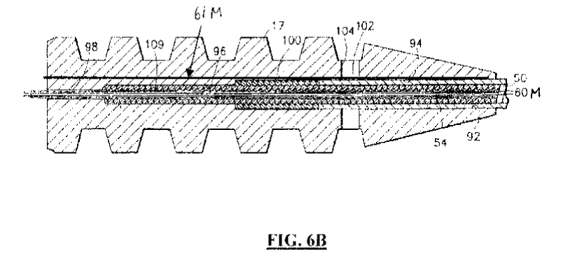

Handle: The construction of the handle 17 is shown in FIGS. 6A& 6B. The proximal end of the shaft 50 and the proximal end of the main ‘control pull wires’ 60M and main ‘deployment pull wires’ 61M extend into handle 17 at the proximal end 16 of the ‘multi forceps biopsy catheter’ 10. The handle 17 comprises a central shaft about which a displaceable spool 19 is disposed. The central shaft has a longitudinally directed stepped diameter bore 92 extending therein, as shown in FIG. 6B. The proximal end of the shaft 50 extends into the bore 92 on the distal end of the central shaft. The bore 92 extending into the central shaft has a three-stepped configuration. The bore 92 on the distal most end of the central shaft has a large first diameter 94; which steps to a smaller second diameter 96; which subsequently steps down to a smaller yet third diameter bore 98 as shown in FIG. 6B. A locking coil 100 is disposed against the first largest diameter bore 94 in the central shaft 50. The shaft 50 has an outer diameter slightly larger than the inner diameter of the locking coil 100 and is threadedly received there through. The shaft 50 thus extends to and abuts the handle 17 adjacent the second stepped bore 96 of the bore 92 in the central shaft. The main control pull wires 60M disposed through the bore 80 of the shaft 50 extend there through and into the smallest portion 98 of the bore 92 in the central shaft. A strain relief sheath 102 is disposed distally to the locking coil 100 about the shaft 50 within the largest bore 94 in the central shaft. The strain relief sheath 102 extends slightly distally of the distal most end of the central shaft, and is bonded to the inner walls of the largest bore 94 by a solvent which is directed thereto through a hole 104, as shown in FIG. 6B. The strain relief sheath 102 limits twist and movement of the shaft 50 with the bore 94 while preventing a sharp bend of the shaft 50 at the distal end of the handle 17. An FEP sheath 54 extends from the distal end of the shaft 50 there through into the central shaft 56 of the handle 17. The sheath 54 acts as a bearing between the main ‘control pull wires’ 60M and the bore 80 of the shaft 50.

The proximal end of the main ‘control pull wires’ 60M extend through the proximal end of the shaft 50 as aforementioned and through and anti-kinking tube 109, and are locked into a cross pin 110, as shown in FIG. 6A, wherein cross pin 110 mates with a slot 112 disposed across the central shaft of the handle 17. The slot 112 is in communication with the axial bore 92 in the central shaft. The proximal most end of the main control pull wires 60M are locked into the cross pin 110 by a setscrew 114 as shown in FIG. 6A. The ends of the cross pins 110 mate with a slot 116 in the spool 19 so as to lock the cross pin 110 therewith. Movement of the spool 19 which is disposed about the central shaft thereby effectuates movement of the main ‘control pull wires’ 60M disposed within the shaft 50, the distal ends of which are attached to the tangs 24 on the jaws 18 as shown in FIGS. 3, 4 &5.

The main ‘deployment pull wires’ 61M extend proximally into the bore of the handle 17; and thereafter extend onto to a deployment actuator 01 located on the handle 17. The deployment actuator 01 comprises of a wheel; and the two main ‘deployment pull wires’ 61M are fixedly attached on the opposite sides of the said wheel. Turning motion of the wheel effects tightening of one main ‘deployment pull wire’ 61M, while loosening the other main ‘deployment pull wire’ 61M. As discussed above; ‘deployment pull wires’ 61 coming from the outer side of the housing 29 of all forceps biopsy assemblies 14 are attached to the same corresponding main ‘deployment pull wire’ 61M. Similarly, ‘deployment pull wires’ 61M coming from the inner side of the housing 29 of all forceps biopsy assemblies 14 are attached to the other corresponding main ‘deployment pull wire’ 61M. In the preferred embodiment, clock-wise movement of the deployment actuator 01 deploys all forceps biopsy assemblies 14 by moving each forceps biopsy assembly 14 away from and perpendicular to the long axis of the shaft 50; as shown in FIG. 5A. Similarly, counter clock movement of the deployment actuator 01 retracts all forceps biopsy assemblies 14 by moving each forceps biopsy assembly 14 towards the shaft 50.

Operation: The operation of the ‘multi forceps biopsy catheter’ is shown in FIGS. 5A &5B. The ‘multi forceps biopsy catheter’ is passed through the instrument channel of the endoscope. The tip of the endoscope is then maneuvered to align the most distal forceps biopsy assembly 14 with the most distal part of the tissue to be sampled. The forceps biopsy modules 14 are then deployed using the deployment actuator 01. In the preferred embodiment of the present invention, clock wise rotation of the deployment actuator 61M deploys the forceps biopsy assemblies by moving the housing 29 of each forceps biopsy assembly perpendicularly and away from the shaft 50; as shown in FIG. 5A. At this time, spool 19 is moved distally relative to the central shaft of the handle 17; thereby opening the jaws 18; as shown in FIG. 5B. We recommend applying negative suction using the air/water channel of the endoscope, which will help bring tissue in close opposition to the jaws 18. The spool 19 is moved proximally relative to central shaft of the handle 17; thereby closing the jaws 18; and whereby a tissue specimen is grasped firmly between the jaws 18 of each such forceps biopsy assembly 14. The ‘multi forceps biopsy catheter’ is then pulled away which severs the grasped tissue from its parent organ. At this time, the jaws 18 are maintained in the closed position, the forceps biopsy assemblies 14 are retracted by counter clock rotation of the deployment actuator 01; and the ‘multi forceps biopsy catheter’ is pulled out of the endoscope. The tissue sample caught between the jaws 18 is then retrieved. The ‘multi forceps biopsy catheter’ provides an effective means to obtain multiple tissue specimens during a single operation of the said catheter; during a single passage through the endoscope. This significantly reduces the time required to complete endoscopic procedures that require multiple tissue specimens be obtained. As per the discussion above, this feature is particularly useful in surveillance practice of Inflammatory Bowel Disease and Barrett's esophagus.

The sideways deployment means of the forceps biopsy assemblies 14 of the ‘multi forceps biopsy catheter’ has an added benefit. With a conventional biopsy catheter, the biopsy catheter and the forceps biopsy assembly contained therein, are oriented parallel to the long axis of the gut lumen during an endoscopic procedure. In order to bring the forceps biopsy assembly in opposition to the tissue to be sampled, the biopsy catheter has to be maneuvered to change its orientation perpendicular to the long axis of the gut lumen; which is usually a difficult maneuver, especially in organs with a narrow lumen, like colon and esophagus. With means provided in the ‘multi forceps biopsy catheter’ to deploy the forceps biopsy assembly 14 perpendicular to the long axis of the biopsy catheter, no additional maneuvering of the biopsy catheter is required to position the forceps biopsy assembly in opposition to the tissue to be sampled. This reduces the time needed to obtain a tissue specimen.

The invention is capable of other embodiments and of being practiced and carried out in various ways. As such, those skilled in the art will appreciate that the conception, upon which this disclosure is based, may readily be utilized as a basis for the designing of other structures, methods and systems for carrying out one or several purposes of the present invention. Some variations of the present inventions are; 1) we have described the biopsy assemblies having forceps mechanism to obtain tissue specimens, however the biopsy assembly may be of other configurations; 2) the operation of the biopsy assemblies in the preferred embodiments of the present invention is manual in nature, however means for electronic operation of the biopsy assemblies can be provided; 3) in the preferred embodiments simultaneous operation of the biopsy assemblies is described, however the biopsy assemblies can be operated sequentially. The list is by no means exhaustive. It is important, therefore, that the claims be regarded as including such equivalent constructions insofar as they do not depart from the spirit and scope of the present invention.

What is claimed is:

1. A biopsy catheter with means to take multiple tissue specimens during a single operation of the said catheter; comprising of a shaft with proximal and distal ends, handle attached to the shaft at its proximal end; the shaft housing one or more biopsy assemblies along its length; and wherein an actuator assembly is provided to operate one or more of the biopsy assemblies.

2. The biopsy catheter of claim 1 wherein; the biopsy assembly comprises of opposed first and second jaws; and a supporting housing on which the first and second jaws are disposed.

3. The biopsy assembly according to claim 2; wherein the housing of the biopsy assembly is attached to the shaft of the biopsy catheter by means of a mechanical articulation; the said housing having a cross pin, wherein said first jaw defines a first pin-receiving hole and said second jaw defines a second pin-receiving hole through which said cross pin extends, and said first and second jaws move about said cross pin.

4. The biopsy assembly of claim 2; wherein an actuator assembly for operating the jaws comprises of pull wires attached to the jaws of the biopsy assembly; extending through one or more bores in the shaft of the biopsy catheter, the said pull wires terminating at an actuator.

5. The actuator assembly of claim 4; wherein the actuator comprises of a spool having a central opening which receives one or more pull wires; and an engagement means in said spool for engaging said pull wires.

6. The actuator assembly of claim 5; wherein movement of the spool results in distal and proximal movement of the pull wires; which in turn results in opening and closing motion respectively of the corresponding jaws of the forceps biopsy assembly.

7. A biopsy catheter comprising of a shaft having a proximal and a distal end; the proximal end having a handle; one or more biopsy assemblies disposed along the length of the shaft; and wherein actuation means is provided to deploy one or more of said biopsy assemblies sideways from the shaft.

8. The biopsy catheter of claim 7; wherein the biopsy assembly comprises of opposed first and second jaws; and a supporting housing on which the first and second jaws are disposed.

9. The biopsy catheter of claim 7; wherein the actuator assembly to deploy one or more biopsy assemblies sideways from the shaft of the biopsy catheter comprises of one or more pull wires attached distally to the housing and proximally to an actuator.

10. The actuator assembly of claim 9; wherein the actuator comprises of a wheel with means to receive one or more pull wires along its perimeter, and wherein rotation of the said wheel causes tension on pull wire disposed on one side and relaxation of pull wire disposed on the opposite side of the wheel.

11. The biopsy catheter of claim 7; wherein actuator assembly for opening and closing of the jaws of the forceps biopsy assembly are provided; and wherein the said actuator assembly for operating the jaws comprises of pull wires attached to the jaws of the biopsy assembly; extending through one or more bores in the shaft of the biopsy catheter, the said pull wires terminating at an actuator assembly on the handle of the biopsy catheter.

12. The actuator assembly of claim 11; wherein the actuator comprises of a spool having a central opening which receives one or more pull wires; and an engagement means in said spool for engaging the said pull wires.

13. The actuator assembly of claim 11; wherein movement of the spool results in distal and proximal movement of the pull wires; which in turn results in opening and closing motion of the corresponding jaws of the forceps biopsy assembly respectively.

14. A method of taking multiple biopsy during a single operation of a biopsy catheter comprising of; 1) a biopsy catheter with multiple biopsy assemblies; 2) wherein one or more of said biopsy assemblies are deployed and operated to take tissue specimens during a single operation of the said biopsy catheter.
